(19) United States
(12) Patent Application Publication
(10) Pub. No.: US 2022/0091118 A1
(43) Pub. Date: Mar. 24, 2022

(54) DETECTION OF CEREBROSPINAL FLUID

(71) Applicants: NSPC Technologies, LLC, Rockville Centre, NY (US); Rensselaer Polytechnic Institute, Troy, NY (US); Gwangju Institute of Science and Technology, Gwangju (KR)

(72) Inventors: Min-Gon Kim, Gwangju (KR); Robert John Linhardt, Albany, NY (US); William J. Sonstein, Old Westbury, NY (US); Jonathan S. Dordick, Schenectady, NY (US); Seok-Joon Kwon, Niskayuna, NY (US); Jusung Oh, Yangsan-si (KR)

(21) Appl. No.: 17/423,939

(22) PCT Filed: Jan. 31, 2020
(86) PCT No.: PCT/US20/16075

§ 371 Date: Jul. 19, 2021

Related U.S. Application Data
(60) Provisional application No. 62/799,943, filed on Feb. 1, 2019.
(60) Provisional application No. 62/799,363, filed on Jan. 31, 2019.

(51) Int. Cl. G01N 33/558; G01N 33/68

(52) U.S. Cl. CPC G01N 33/558; G01N 2333/79; G01N 2333/42; G01N 33/68

(57) ABSTRACT

The invention encompasses methods and test strips for detecting the presence of cerebrospinal fluid (CSF) in a biological sample comprising removing sialo-transferrin and selectively detecting or measuring asialo-transferrin in the biological sample.

### CROSS-REFERENCE TO RELATED APPLICATIONS

This application claims benefit of U.S. Provisional Application No. 62/799,363, filed Jan. 31, 2019, and claims benefit of U.S. Provisional Application No. 62/799,943 filed Feb. 1, 2019, the contents of each of which are hereby incorporated by reference.

### BACKGROUND OF THE INVENTION

Spinal fluid leak as a result of incidental durotomy during spinal surgery is a relatively common complication that occurs with an incidence of 2-17% [1-6]. Usually, spinal fluid leaks are recognized at the time of surgery and are successfully repaired. Occasionally, they present in a delayed fashion, for example, if a small durotomy is not recognized at the time of surgery or if the repair is not ideal initially. Spine surgeons are frequently confronted with post-operative fluid collections that may or may not represent a CSF (cerebrospinal fluid) leak. This is more commonly an issue with lumbar spine surgery for degenerative disease. If a patient presents with positional headaches or with clear fluid leakage, then the diagnosis is more easily made. However, in the post-operative period it is sometimes confounding differentiating seromatous fluid from CSF as a patients' symptoms do not always classically present. A patient may present with a bulging subcutaneous collection of fluid whereupon aspiration, the nature of the fluid is not certain. In surgical decision-making, it would be ideal to confirm the diagnosis of CSF leak quickly so that one can initiate repair, which requires surgical intervention particularly if there is skin drainage, which could result in meningitis. It would be advantageous to know if the collection is a seroma as these can often be treated conservatively without return to the operating room. Currently to distinguish CSF from seromatous fluid, one must send out the fluid sample to a laboratory utilizing electrophoresis and obtaining the results can take three to five days.

A combination of protein separation and detection, using electrophoresis and mass spectrometry, has been successfully applied to identify protein biomarkers in CSF [7]. Transferrin (TF) isoforms among protein biomarkers in CSF have been used as a critical diagnostic marker not only for detecting CSF leakage from liquorrhea but also detecting several diseases, including early stage oral cancer [8], chronic alcoholism [9], and diabetic kidney disease [10]. Transferrin (TF) is a glycoprotein important for maintaining human iron homeostasis. TF is modified to β2TF (asialo-transferrin) in the CSF through the action of brain neuraminidase resulting in the elimination of terminal sialic acid residues on the glycan chains of TF, affording the β2TF glycoform constituting up to 30% of total CSF transferrin. Hence, sensitive and reliable detection of β2TF in non-CSF body fluid samples can point to CSF leakage.

However, although the detection of β2TF has been used in the diagnosis of CSF leakage, there remain several practical limitations in using this method for a point-of-care diagnosis. The minor differences in the TF-based glycan chains make it difficult to distinguish β2TF from sialo transferrin (sTF) since sTF is also a major component in serum, thus sensitivity and specificity are very important. Currently, these TF glycoforms are distinguished using electrophoresis, requiring a relatively long processing time (120-150 min) and requires analysis by skilled professionals for diagnosis of CSF leakage. Moreover, an electrophoresis-based assay is usually performed in remote highly specialized professional clinical laboratories that requires additional turnaround time for sample analysis. Thus, conventional electrophoresis for detecting β2TF is not actually suitable as a POC assay for rapid diagnosis and immediate treatment of CSF leakage—which can be critical for patient health.

There remains a need in the art for simple methods and devices for the near real-time rapid detection of CSF leakage which can be readily employed by medical staff during surgical procedures.

### SUMMARY OF THE INVENTION

Herein disclosed is a novel rapid, sensitive and specific assay for the determination of β2TF in fluids, useful in the diagnosis of CSF leakage.

A product for selectively detecting asialo-transferrin in a biological sample comprising:

A) a lateral flow device comprising in sequential order:
a portion comprising a first plurality of transferrin-binding antibodies conjugated to nanoparticles but not conjugated to the lateral flow device itself;
a portion comprising a fixed sialic acid-specific lectin; and
a portion comprising a second plurality of transferrin-binding antibodies affixed to the lateral flow device; and
a portion comprising a plurality of anti-antibody antibodies affixed to the lateral flow device,
   or
   B) a first plurality of transferrin-binding antibodies conjugated to nanoparticles but not conjugated to a lateral flow device;
   and a lateral flow device comprising in sequential order:
a portion comprising a fixed sialic acid-specific lectin;
a portion comprising a second plurality of transferrin-binding antibodies affixed to the lateral flow device; and
a portion comprising a plurality of anti-antibody antibodies affixed to the lateral flow device.

A kit comprising:

i) the lateral flow device as described herein and
ii) a container comprising the first plurality of transferrin-binding antibodies conjugated to nanoparticles.

A method of detecting asialo-transferrin in a biological sample comprising:

a) contacting the biological sample with the first plurality of transferrin-binding antibodies conjugated to nanoparticles of part B) as described herein;
b) centrifuging the product of step a) so as to separate and obtain conjugates of transferrin bound to transferrin-binding antibodies conjugated to nanoparticles;
c) contacting the conjugates obtained in step b) with the lateral flow device as described herein and observing if asialo-transferrin bound antibodies bind to the second plurality of transferrin-binding antibodies affixed to the lateral flow device of B), wherein if such antibodies bind then asialo-transferrin has been detected in the biological sample and wherein if no asialo-transferrin bound antibodies bind to the second plurality of transferrin-binding antibodies affixed to the lateral flow device then asialo-transferrin has not been detected in the biological sample.

A method of detecting asialo-transferrin in a biological sample comprising:

a) contacting the biological sample with the first plurality of transferrin-binding antibodies conjugated to nanoparticles of part A) as described herein so as obtain conjugates of transferrin bound to transferrin-binding antibodies conjugated to nanoparticles;
b) contacting the conjugates obtained in step a) with the lateral flow device of as described herein and observing if asialo-transferrin bound antibodies bind to the second plurality of transferrin-binding antibodies affixed to the lateral flow device of A), wherein if such antibodies bind then asialo-transferrin has been detected in the biological sample and wherein if no asialo-transferrin bound antibodies bind to the second plurality of transferrin-binding antibodies affixed to the lateral flow device then asialo-transferrin has not been detected in the biological sample.

A method comprising:

performing surgery on the central nervous system of a subject;

obtaining one or more samples of the subject's blood, wherein if more than one sample is obtained then the samples are obtained at different time points during the surgery; and

detecting if cerebrospinal fluid has leaked into the blood of the subject during surgery comprising contacting the lateral flow device as described herein with the one or more blood samples and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

A method comprising:

performing surgery on the central nervous system of a subject;
obtaining one or more samples of the subject's blood, wherein if more than one sample is obtained then the samples are obtained at different time points during the surgery; and
detecting if cerebrospinal fluid has leaked into the blood of the subject during surgery comprising contacting the sample with the first plurality of transferrin-binding antibodies conjugated to nanoparticles but not conjugated to the lateral flow device itself of part B) with the one or more blood samples, centrifuging the product thereof so as to separate and obtain conjugates of transferrins bound to transferrin-binding antibodies conjugated to nanoparticles, contacting the conjugates obtained with the lateral flow device as described herein and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

A lateral flow device comprising, in sequential order, a portion comprising a fixed sialic acid-specific lectin; a portion comprising a plurality of transferrin-binding antibodies affixed to the lateral flow device; and a portion comprising a plurality of anti-antibody antibodies affixed to the lateral flow device.

A lateral flow device is provided for selectively detecting asialo-transferrin in a biological sample comprising in sequential order:

### A)

a portion comprising a first plurality of transferrin-binding antibodies;
one or more portions each comprising a fixed sialic acid-specific lectin; and
a portion comprising a second plurality of transferrin-binding antibodies, or

### B)

a portion comprising a fixed sialic acid-specific lectin;
one or more portions each comprising a first plurality of transferrin-binding antibodies; and
a portion comprising a second plurality of transferrin-binding antibodies.

Also provided is a method of detecting asialo-transferrin in a biological sample comprising contacting the lateral flow device as described herein with the sample and observing if asialo-transferrin bound antibodies bind to the second plurality of transferrin-binding antibodies, wherein if such antibodies bind then asialo-transferrin has been detected in the biological sample and wherein if no asialo-transferrin bound antibodies bind to the second plurality of transferrin-binding antibodies then asialo-transferrin has not been detected in the biological sample.

Also provided is a method of detecting cerebrospinal fluid in a biological sample comprising contacting the lateral flow device as described herein with the sample and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

Also provided is a method comprising:

performing surgery on the central nervous system of a subject;
obtaining one or more samples of the subject's blood, wherein if more than one sample is obtained then the samples are obtained at different time points during the surgery; and detecting if cerebrospinal fluid has leaked into the blood of the subject during surgery comprising contacting the lateral flow device as described herein with the one or more blood samples and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A-1C: Schematic illustration of detection strategy of beta-2 transferrin (β2TF) for determination of cerebrospinal fluid (CSF) leakage. (1A) Sample pretreatment process, (1B) structure of immunochromatographic assay (ICA), and (1C) reaction mechanisms of the entire process.

FIG. 2A-2B: Demonstration of specificity of lectin in immunochromatographic assay (ICA) by evaluation of (2A) oxidation effect of antibody. (2B) Comparison of colorimetric signal intensity between sialo-transferrin (sTF), beta-2 transferrin (β2TF), and control.

FIG. 3A-3C: (3A) Evaluation of efficiency of deletion lines, sensitivity of test lines and effect of sample pretreatment process (C stands for control line, T: test line, and D: deletion lines), (3B) the comparison of sum of colorimetric signal intensity of deletion lines, and (3C) signal intensity of test line.

FIG. 4A-4B: Performance of immunochromatographic assay (ICA) used for mixed sample based on (4A) cerebrospinal fluid (CSF) proportion and (4B) calculated value (the signals of a test line divided by the signals of the sum of deletion lines) using test and deletion lines.

FIG. 5: Comparison of calculated value between clinical samples (positive n=13, negative n=34, artificial mixture=16, *** stands for p value<0.001, ** for <0.01)

FIG. 6: Receiver operating characteristic (ROC) analysis of clinical samples based on parameter of immunochromatographic assay (ICA) for determination of cerebrospinal fluid (CSF) leakage.

### DETAILED DESCRIPTION OF THE INVENTION

Herein is disclosed a method and devices for detection of β2TF using an immunochromatographic assay (ICA). Sialic acid-specific lectin is immobilized into multiple deletion lines at the beginning of a test strip to capture sTF, and anti-transferrin antibodies are immobilized in a detection line near the end of the test strip. Thus, the serum-specific sTF is selectively captured early in the deletion lines, allowing β2TF to move alone along the test strip to the detection line. In addition to the enhanced efficiency of sTF capture by sialic acid-specific lectin, optional pre-treatment process step(s) eliminates the binding of other unidentified sialo-glycoproteins and also reduced the “hook effect,” which otherwise results in a higher likelihood for false negatives despite high concentrations of analyte. The method(s) and the device(s) result in determination of CSF in test samples with as good as 97.1% or more specificity and 96.2% or more sensitivity.

A product for selectively detecting asialo-transferrin in a biological sample comprising:

A) a lateral flow device comprising in sequential order:
a portion comprising a first plurality of transferrin-binding antibodies conjugated to nanoparticles but not conjugated to the lateral flow device itself;
a portion comprising a fixed sialic acid-specific lectin; and
a portion comprising a second plurality of transferrin-binding antibodies affixed to the lateral flow device; and
a portion comprising a plurality of anti-antibody antibodies affixed to the lateral flow device,
   or
   B) a first plurality of transferrin-binding antibodies conjugated to nanoparticles but not conjugated to a lateral flow device;
   and a lateral flow device comprising in sequential order:
a portion comprising a fixed sialic acid-specific lectin;
a portion comprising a second plurality of transferrin-binding antibodies affixed to the lateral flow device; and
a portion comprising a plurality of anti-antibody antibodies affixed to the lateral flow device.

In embodiments, the lateral flow device comprises A). In embodiments, the lateral flow device comprises B). In embodiments, the lateral flow device comprises a nitrocellulose membrane.

In embodiments, the first and second pluralities of transferrin-binding antibodies are IgG antibodies and/or wherein the plurality of anti-antibody antibodies is a plurality of anti-IgG antibodies. In embodiments the plurality of anti-antibody antibodies are anti-antibodies of the type of antibody that the first and second pluralities of transferrin-binding antibodies are. For example, if the first and second pluralities of transferrin-binding antibodies are goat, rabbit or human antibodies then the anti-antibody antibodies are anti-goat antibody antibodies, anti-rabbit antibody antibodies, or anti-human antibody antibodies, respectively. Preferably, the plurality of anti-antibody antibodies acts as a control line in the device, e.g. confirming fluid sample movement through the device.

In embodiments, each antibody of the second plurality of transferrin-binding antibodies is conjugated to a nitrocellulose membrane of the lateral flow device, and/or wherein the sialic-specific lectin is affixed to a nitrocellulose membrane of the lateral flow device.

In embodiments, multiple antibodies of the first plurality of transferrin-binding antibodies conjugated to nanoparticles are conjugated to the same nanoparticle.

In embodiments, the nanoparticles comprise spherical gold nanoparticles. In embodiments, the nanoparticles comprise latex microspheres.

In embodiments, the device further comprises a fluid sample pad prior in sequential order to (i) the portion comprising a first plurality of transferrin-binding antibodies of A), or to (ii) the portion comprising a fixed sialic acid-specific lectin of B).

In embodiments, the lectin comprises a Sambucus nigra lectin. In an embodiment, the lectin is biotinylated. In an embodiment, the lectin is not biotinylated.

In embodiments, the device further comprises a fluid-absorbent pad subsequent in sequential order to the portion comprising a plurality of anti-antibody antibodies.

In embodiments, the sialic acid residues on glycan chains of the transferrin-binding antibodies have been oxidized.

In embodiments, the transferrin-binding antibodies which have had their sialic acid residues oxidized show reduced binding to sialic acid-specific lectin compared to transferrin-binding antibodies which have not had their sialic acid residues oxidized.

In embodiments, the transferrin-binding antibodies have been oxidized by treating them with a periodate.

In embodiments, the portion comprising a plurality of anti-antibody antibodies affixed to the lateral flow device is a control line.

A kit comprising:

i) the lateral flow device as described herein and
ii) a container comprising the first plurality of transferrin-binding antibodies conjugated to nanoparticles.

In embodiments, the sialic acid residues on glycan chains of the first plurality of transferrin-binding antibodies have been oxidized.

In embodiments, the transferrin-binding antibodies which have had their sialic acid residues oxidized show reduced binding to sialic acid-specific lectin compared to transferrin-binding antibodies which have not had their sialic acid residues oxidized.

In embodiments, the nanoparticles comprise gold nanoparticles.

A method of detecting asialo-transferrin in a biological sample comprising:

a) contacting the biological sample with the first plurality of transferrin-binding antibodies conjugated to nanoparticles as described herein;
b) centrifuging the product of step a) so as to separate and obtain conjugates of transferrin bound to transferrin-binding antibodies conjugated to nanoparticles;
c) contacting the conjugates obtained in step b) with the lateral flow device as described herein, and observing if asialo-transferrin bound antibodies bind to the second plurality of transferrin-binding antibodies affixed to the lateral flow device of B), wherein if such antibodies bind then asialo-transferrin has been detected in the biological sample and wherein if no asialo-transferrin bound antibodies bind to the second plurality of transferrin-binding antibodies affixed to the lateral flow device then asialo-transferrin has not been detected in the biological sample.

A method of detecting asialo-transferrin in a biological sample comprising:

a) contacting the biological sample with the first plurality of transferrin-binding antibodies conjugated to nanoparticles as described herein so as obtain conjugates of transferrin bound to transferrin-binding antibodies conjugated to nanoparticles;
b) contacting the conjugates obtained in step a) with the lateral flow device as described herein and observing if asialo-transferrin bound antibodies bind to the second plurality of transferrin-binding antibodies affixed to the lateral flow device of A), wherein if such antibodies bind then asialo-transferrin has been detected in the biological sample and wherein if no asialo-transferrin bound antibodies bind to the second plurality of transferrin-binding antibodies affixed to the lateral flow device then asialo-transferrin has not been detected in the biological sample.

In embodiments, the method further comprises determining if the portion comprising a plurality of anti-antibody antibodies affixed to the lateral flow device has anti-transferrin antibody bound thereto after the sample has been contacted with the device.

In embodiments, the method further comprises obtaining the sample from a subject, prior to step a).

In embodiments, the subject is undergoing, or has previously undergone, surgery.

In embodiments, the surgery is a neurological surgery.

A method comprising:

performing surgery on the central nervous system of a subject;
obtaining one or more samples of the subject's blood, wherein if more than one sample is obtained then the samples are obtained at different time points during the surgery; and

detecting if cerebrospinal fluid has leaked into the blood of the subject during surgery comprising contacting the lateral flow device as described herein with the one or more blood samples and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

A method comprising:

performing surgery on the central nervous system of a subject;
obtaining one or more samples of the subject's blood, wherein if more than one sample is obtained then the samples are obtained at different time points during the surgery; and
detecting if cerebrospinal fluid has leaked into the blood of the subject during surgery comprising contacting the sample with the first plurality of transferrin-binding antibodies conjugated to nanoparticles but not conjugated to the lateral flow device itself as described herein with the one or more blood samples, centrifuging the product thereof so as to separate and obtain conjugates of transferrins bound to transferrin-binding antibodies conjugated to nanoparticles, contacting the conjugates obtained with the lateral flow device as described herein and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

In embodiments, the sample is a serum sample, an otorrhea sample, a rhinorrhea sample, or comprises drainage from a spinal suture area.

In embodiments, centrifuging the product of step a) so as to separate and obtain conjugates of transferrin bound to transferrin-binding antibodies conjugated to nanoparticles separates the conjugates from all, or substantially all, non-transferrin sialyated glycoproteins from the sample.

In embodiments, the method further comprises oxidizing sialic acid residues on glycan chains of the first plurality of transferrin-binding antibodies prior to contacting with the sample.

A lateral flow device comprising, in sequential order, a portion comprising a fixed sialic acid-specific lectin; a portion comprising a plurality of transferrin-binding antibodies affixed to the lateral flow device; and a portion comprising a plurality of anti-antibody antibodies affixed to the lateral flow device.

In embodiments, the plurality of transferrin-binding antibodies comprises IgG antibodies and/or wherein the plurality of anti-antibody antibodies comprises a plurality of anti-IgG antibodies.

In embodiments, each antibody of the plurality of transferrin-binding antibodies is conjugated to a nitrocellulose membrane of the lateral flow device, and/or wherein the sialic-specific lectin is affixed to a nitrocellulose membrane of the lateral flow device.

In embodiments, the device further comprises a fluid sample pad prior in sequential order to the portion comprising a fixed sialic acid-specific lectin.

In embodiments, the device further comprises a fluid-absorbent pad subsequent in sequential order to the portion comprising a plurality of anti-antibody antibodies.

In embodiments, the sialic acid residues on glycan chains of the transferrin-binding antibodies have been oxidized.

In embodiments, the transferrin-binding antibodies which have had their sialic acid residues oxidized show reduced binding to sialic acid-specific lectin compared to transferrin-binding antibodies which have not had their sialic acid residues oxidized.

In embodiments, the transferrin-binding antibodies have been oxidized by treating them with a periodate. In embodiments of the devices and methods, the periodate comprises sodium metaperiodate.

In embodiments, the portion comprising a plurality of anti-antibody antibodies affixed to the lateral flow device is a control line.

In embodiments, observing binding is performed by observing a color change on the device in the portion comprising the second plurality of transferrin-binding antibodies affixed to the lateral flow device.

As used herein, the words “a” and “an” are meant to include one or more unless otherwise specified. For example, the term “a particle” encompasses one or more particles.

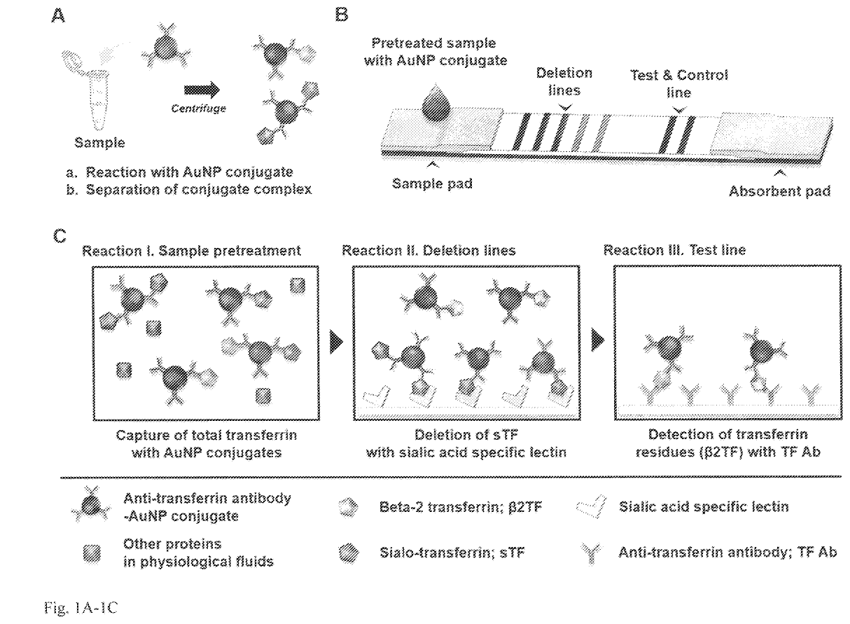

Transferrin (TF) is a secreted glycoprotein, having multiple glycoforms, containing glycans capped at their non-reducing ends with negatively charged sialic acid residues [11, 12]. TF plays a crucial role in homeostasis and transport of iron, as well as in protecting the body against free radical damage associated with unbound iron [13]. TF in serum is composed of 679 amino acid residues (˜78 kDa MW) and has two glycosylation sites at asparagine Asn432 and Asn630 that are often occupied by N-linked glycans harboring various number of terminal (non-reducing end) sialic acid (or N-acetylneuraminic acid) residues, resulting in a heterogeneous populations of TF glycoforms [11, 14] (FIGS. 1A and 1B). TF in serum is exclusively comprised of fully sialylated glycoforms. In contrast, TF in CSF, referred to as β2-transferrin (β2TF), exists as a mixture of sialo (sTF) and asialoglycoforms (aTF) [7, 12] (FIGS. 1A and 1B). It has been speculated that the aTF in CSF originates from serum sTF through the action of brain neuraminidase [15]. Over the years, a number of methods have been designed to detect TF isoforms as biomarkers of CSF leakage, as well as various disorders of the central nervous system [16, 17]. Different separation methods relying on electrophoresis have been developed to separate TF isoforms, including isoelectric focusing [18], immunofixation gel electrophoresis [19], sodium dodecyl sulfate-polyacrylamide gel electrophoresis (SDS-PAGE) [20], and capillary electrophoresis (CE) [21].

The present invention encompasses a simple and rapid method enabling a spine surgeon to detect and/or measure CSF directly from a biological sample, such as a post-operative drainage. Spine surgeons can be faced with critical, time-sensitive decisions regarding patient care when a fluid leak is detected at the surgical site of a patient's postoperative incision.

In some embodiments, the invention specifically excludes the use of chemical and/or enzymatic methods to specifically oxidize sTF in a biological sample, such as serum, allowing it to be conjugated to a hydrazide reagent (such as hydrazide magnetic microparticles) and selectively removed from the sample, allowing the rapid detection of CSF-derived aTF by a method amenable to use in rapid, real-time “dip-stick” analysis.

The biological sample can be any sample suspected of containing CSF, transferrin, and/or an asialo-transferrin. Exemplary biological samples include, for example, serum, blood, plasma, nasal fluid, aural fluid, a biopsy sample, a lymphatic fluid sample, fluid from a head or spinal wound or puncture, and fluid from a surgical incision site. In certain embodiments, the biological sample is serum. In additional embodiments, the biological sample is obtained from a subject, such as a human patient, during or after surgery. In yet additional embodiments, the biological sample is obtained from a surgical incision site or a post-operative fluid collection. The term “subject” is mean to encompass an animal subject including, but not limited, a human subject. In certain embodiments, the biological sample is obtained from a human subject or is of human origin.

In sialo-transferrin (sTF), a terminal residue is a sialic acid residue. When the sialic acid groups are removed from sTF, the terminal monosaccharide residue is galactose.

TF in CSF exists as a mixture of sialo (sTF) and asialo-transferrin (aTF). In contrast, in serum, the transferrin is exclusively comprised of fully sialylated glycoforms. Removal of sTF from the biological sample will allow the detection and measurement of asialo-transferrin. Because asialo-transferrins are found in CSF (and not normally found in serum), detecting or measuring asialo-transferrin in the biological sample is indicative of a CSF leak. However, due to the concerns and consequences of neurological surgery and CSF leaks, it is imperative to detect such leaks as quickly as possible, or monitor for them during surgery with rapid and sensitive feedback. In addition, given the small amounts of CSF leaked relative to blood volume, the aTF must be detectable, over noise, at very low levels.

A lateral flow device is provided for selectively detecting asialo-transferrin in a biological sample comprising in sequential order:

### A)

a portion comprising a first plurality of transferrin-binding antibodies;
one or more portions each comprising a fixed sialic acid-specific lectin; and
a portion comprising a second plurality of transferrin-binding antibodies, or

### B)

a portion comprising a fixed sialic acid-specific lectin;
one or more portions each comprising a first plurality of transferrin-binding antibodies; and
a portion comprising a second plurality of transferrin-binding antibodies.

In embodiments, the lateral flow device comprises an immunoassay strip or an immunochromatography assay, or a test strip. In embodiments, the lateral flow device works primarily along a single axis, e.g. in a test strip format.

In embodiments, the device further comprises a portion comprising a plurality of anti-antibody antibodies, which portion is subsequent in order to the portion comprising a second plurality of transferrin-binding antibodies. In embodiments, the portion comprising a plurality of anti-antibody antibodies is a control zone or control line.

In embodiments, the first and second pluralities of transferrin-binding antibodies are IgG antibodies and wherein the plurality of anti-antibody antibodies is a plurality of plurality of anti-IgG antibodies.

In embodiments, each antibody of the first plurality of transferrin-binding antibodies is conjugated to a gold nanoparticle. In embodiments, each antibody of the first plurality of transferrin-binding antibodies is conjugated to a latex microsphere.

In embodiments, multiple antibodies of the first plurality of transferrin-binding antibodies are conjugated to the same gold nanoparticle. In embodiments, multiple antibodies of the first plurality of transferrin-binding antibodies are conjugated to the same latex microsphere.

In embodiments, the second plurality of transferrin-binding antibodies is affixed to a solid support. In embodiments, the portion comprising a second plurality of transferrin-binding antibodies affixed to a solid support is a test zone or test line.

In embodiments, the device further comprises a fluid sample pad prior in sequential order to A) or B). In embodiments, the fluid sample pad comprises an adsorbent pad onto which the test sample is applied.

In embodiments, the device further comprises a fluid-absorbent pad subsequent in sequential order to the portion comprising a plurality of anti-antibody antibodies. In embodiments, the fluid-absorbent pad can comprise a wick or waste reservoir to draw the fluid of the sample across a reaction membrane by capillary action and, optionally, collect it.

In embodiments, the device comprises a reaction membrane. In embodiments, the reaction membrane is a nitrocellulose or cellulose acetate membrane onto which anti-transferrin analyte antibodies are immobilized in a line that crosses the membrane to act as a capture zone or test line. In embodiments, a control zone is also present containing antibodies specific for the conjugate antibodies.

In embodiments, one or more of the recited components of the lateral flow device/strip are fixed to an inert backing material. In embodiments, the lateral flow device has a dipstick format. In embodiments, the lateral flow device has a plastic casing with a sample port and reaction window showing capture and control zones.

Also provided is a method of detecting asialo-transferrin in a biological sample comprising contacting the lateral flow device as described herein with the sample and observing if asialo-transferrin bound antibodies bind to the second plurality of transferrin-binding antibodies, wherein if such antibodies bind then asialo-transferrin has been detected in the biological sample and wherein if no asialo-transferrin bound antibodies bind to the second plurality of transferrin-binding antibodies then asialo-transferrin has not been detected in the biological sample.

Also provided is a method of detecting cerebrospinal fluid in a biological sample comprising contacting the lateral flow device as described herein with the sample and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

In embodiments, the method further comprises obtaining the sample from a subject.

In embodiments, the subject is undergoing, or has previously undergone, surgery.

In embodiments, the surgery is a neurological surgery.

Also provided is a method comprising:

performing surgery on the central nervous system of a subject;
obtaining one or more samples of the subject's blood, wherein if more than one sample is obtained then the samples are obtained at different time points during the surgery; and
detecting if cerebrospinal fluid has leaked into the blood of the subject during surgery comprising contacting the lateral flow device as described herein with the one or more blood samples and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

In certain aspects of the invention, the sample is diluted after it is obtained and prior to application to the lateral flow device.

The methods herein require a centrifugation pretreatment step to remove interfering sialo-glycoproteins in the sample competing with sTF for the sialic acid-binding lectin immobilized in the deletion lines.

As will be understood by those of skill in the art, once detected, the amount of transferrin or residual transferrin in the sample can be measured using standard curves.

In some aspects, an anti-transferrin antibody is a polyclonal antibody, a monoclonal antibody, or a transferrin-binding fragment of an antibody such as a Fab fragment. Anti-transferrin antibodies can be prepared using convention methods well known to skilled artisans such as methods set forth in Harlow, E., Using Antibodies: A Laboratory Manual, Cold Spring Harbor Laboratory Press, Cold Spring Harbor, N.Y., 1999, or can be purchased from a commercial supplier. In some embodiments, the methods and test strips described herein comprise the use of two antibodies (for example, an antibody in the binding region and an antibody in the capture region of a test strip). The two antibodies can be of different types, for example, the first antibody can be a mouse monoclonal antibody and the second antibody can be a rabbit polyclonal antibody, or vice versa, or the antibodies can bind to different epitopes of transferrin.

In additional embodiments, an anti-transferrin antibody is followed by the addition of a labelled detection antibody to the solid support. In some embodiments, the labelled detection antibody is a labelled anti-transferrin antibody. The labelled detection antibody can, for example, be an antibody conjugated to a detectable label including, for example, a fluorogenic label, a chromogenic label, a biotin molecule, and/or a gold particle. In certain aspects, the labelled detection antibody is a biotinylated antibody which can be detected by adding streptavidin-peroxidase complex, removing unbound conjugates, and adding a peroxidase substrate, such as TMB (3,3′,5,5′-tetramethylbenzidine) or ABTS (2,2′-azino-bis(3-ethylbenzothiazoline-6-sulphonic acid)).

In some embodiments, the test strip comprises a sample loading region and binding region downstream of the sample loading region, wherein the binding region comprises an anti-transferrin antibody. In some aspects, the anti-transferrin antibody in the binding region is a labeled antibody. Transferrin is detected and/or measured as the labelled anti-transferrin antibody become visible or is otherwise detected. In additional aspects, the test strip further comprises a capture region downstream of the binding region, wherein the capture region comprises a capture reagent that binds the anti-transferrin antibody-transferrin complex. In yet additional embodiments, the capture reagent is immobilized in the test strip. In further aspects, the capture reagent is an antibody. In yet further embodiments, the test strip additionally comprises a control region comprising a control reagent. A control reagent can, for example, be an antibody with binding affinity for the labelled antibody.

The invention also encompasses a method of detecting a CSF leak in a subject during or after surgery comprising: a. obtaining a first biological sample from the subject prior to surgery and measuring the asialo-transferrin present in the first sample; and b. obtaining a second biological sample from the subject during or after surgery and measuring the transferrin present in the second sample; wherein a higher amount of asialo-transferrin present in the second sample compared to the asialo-transferrin present in the first sample indicates a CSF leak.

In exemplary methods, a test sample is added to the lateral flow device, e.g. a sample pad thereof, typically followed by a chase buffer. The chase buffer facilitates the flow of fluids across the test surface. The test strip also contains (optionally labelled) antibodies, such as gold particles attached to antibodies. The transferrin present in the sample can bind to the (labeled) antibodies and the complex migrates through the membrane by capillary action. The analyte and label complex can then bind to antibodies which are immobilized on the membrane, creating a detectable indicator, such as a colored line, in the test zone. If no analyte is present in the sample, then the conjugate migrates past the test zone and will not bind to the antibodies on the test line of the membrane. Optionally, a control reagent can capture and bind excess conjugate. In some embodiments, a control reagent and line produced therefrom is a control that indicates the test was run properly. In some embodiments, the results can be read in about 1 to about 60 minutes, or in about 1 minute to about 30 minutes, or in about 5 to about 15 minutes.

In yet additional embodiments, the invention is a device for detecting the presence of transferrin in a sample, wherein the device comprises a test strip described herein and a housing containing the test strip, wherein the housing comprises at least one opening to expose the surface of the test strip in the sample loading zone to the sample. In some embodiments, the device is a handheld device.

Various embodiments described herein involve the use of labelled antibodies. Exemplary labels include, for example, enzymes and their resultant effects on a substrate, colloidal metal particles, latex with dye incorporated, and dye particles. An enzyme can react on a substrate to produce a product that is detectable, for example, by color of absorption (e.g., ultraviolet, visible, infrared), or by fluorescence. In yet additional embodiments, the label is a fluorogenic label, a chromogenic label, a biotin molecule, and/or a metal particle. In some aspects, the metal particles can comprise platinum, gold, silver, selenium, or copper or any other of metal compounds which exhibit characteristic colors. The metal particles suitable for use in the present invention can be prepared by conventional methodologies. For example, the preparation of gold sol particles is described Frens, Nature 241: 20-22 (1973).

In further embodiments, the test strip comprises a solid support (plastic, cardboard, or other rigid or semi-rigid material, and a membrane on top of the solid support (in some examples, the membrane is a nitrocellulose or PVDF membrane). The membrane includes the sample loading region and binding region as described herein. The membrane can also include the capture region and/or control region.

In some embodiments, the transferrin is β2-transferrin, for example, asialo-transferrin. In certain aspects, the sample is a biological sample. In yet additional embodiments, the invention is a method of detecting the presence of CSF in biological sample comprising contacting the sample with a device described herein, wherein the method comprises detecting or measuring the transferrin in the binding region or capture region or downstream of the binding region or capture region.

The invention is illustrated by the following examples which are not meant to be limiting in any way.

### EXEMPLIFICATION

A scheme and structure for a device of the invention, effecting sialo-transferrin selective detection, and asialo-transferrin detection, for diagnosis of cerebrospinal leakage is set forth in FIG. 1. The device can be a lateral flow immunoassay device, for example. The biological sample is applied to the sample pad and is drawn, or wicks, laterally along the device.

Materials

Surfactant 10G (95R-103) and bovine serum albumin (BSA) were from Fitzgerald Industries International (Acton, Mass., USA). Anti-transferrin monoclonal antibody (4T15-8B9; conjugated Ab, 4T15-11D3; immobilized Ab) was obtained from Hytest (Joukahaisenkatu, Turku, Finland). Neo protein saver (NPS-301) was obtained from Toyobo (Satte City, Kamiyoshiba, Japan). Spin column tubes (69725), and spin desalting columns (89891) were purchased from Thermo Fisher Scientific (Waltham, Mass., USA). G1 reaction buffer (B1723) was from New England Biolabs (Ipswich, Mass., USA). Sambucus nigra lectin (L-1300) and biotinylated S. nigra lectin (B-1305) were from Vector laboratories (Burlingame, Calif., USA). Centrifugal filters (UFC510096), laminated cards (HF000MC100) and the nitrocellulose (NC) membrane (HFB01804) were from Millipore (Billerica, Mass., USA). Sample and absorbent pads (Grade 222) were sourced from Bore da Biotech (Seongnam-si, Gyeonggi-do, Korea). Gold colloidal solution was from BBI International (EM.GC20; Cardiff, UK). Polyvinylpyrrolidone (PVP 29K), transferrin (T8158), neuraminidase (N2876), human serum (H4522), sodium metaperiodate (S1878), streptavidin (S4762), and other chemicals were from Sigma-Aldrich (St. Louis, Mo. USA). All buffers and reagent solutions were prepared using distilled water generated using an ELGA water purification system (Lane End, UK).

Antibody Oxidation

Sialic acid residues on the glycan chains of the anti-transferrin antibody were oxidized to reduce antibody binding to the sialic acid-specific lectin. Antibody (1 mg mL⁻¹) was treated with 1 mM sodium metaperiodate in acetate buffer solution (0.1 M, pH 5.5). After incubation at 4° C. for 30 min, the sodium metaperiodate was removed using desalting resin-based centrifugation at 1,000×g for 3 min at 4° C. The desalting resin was prepared in a spin column tube with 750 μL after washing with 1×phosphate-buffered saline (PBS) and centrifugation three times at 1,000×g for 3 min at 4° C. After desalting, the diluted antibody solution was replaced by washing with 1×PBS using a centrifugal filter at 12,000×g for 20 min at 4° C. The antibody was then bound to BSA (Mr=66 kDa) using a BSA antibody ratio of 1:10 for 90 min at 25° C. After the BSA treatment, unbound BSA was removed by centrifugal ultrafiltration (MWCO 100 kDa) with 1×PBS at 12,000×g for 20 min at 4° C. After filtration, the antibody-BSA complex was diluted with 1×PBS to 1 mg mL⁻¹ based on the initial amount of antibody. This treated antibody is designated as “oxidized-antibody.”

Preparation of Streptavidin-Gold Nanoparticle (AuNP) Conjugate

Streptavidin (10 μL, 1 mg mL⁻¹) was added to a mixture of 1 mL of 20 nm colloidal gold nanoparticles (AuNP, 1 OD) and 100 μL of borate buffer (0.1 M, pH 8.4). After incubation at room temperature (RT, 25° C.) for 30 min, 100 μL Neo protein saver (50 mg mL⁻¹) was added to this mixture to block the residual sites on the surface of the AuNPs. After incubation at 4° C. for 60 min, the mixture was centrifuged using a refrigerated micro centrifuge (Smart R17; Hanil Science Industrial Co., Gangwon-do, Korea) at 13,475×g for 20 min at 10° C. The supernatant was discarded, the AuNP conjugates were re-suspended in 10 mM borate buffer (pH 8.4), and the centrifugation and re-suspension steps were repeated twice. The final re-suspended AuNP conjugate solution was concentrated 2-fold by changing the solution volume to 1×PBS containing 50 mg mL⁻¹ trehalose, 5 mg mL⁻¹ neo protein saver, 2 mg mL⁻¹ Tween 20, and 10 mg mL⁻¹ Triton-X 100. Prior to using the AuNP-streptavidin conjugate, the solution was diluted with the same volume of 1×PBS solution.

Preparation of ICA Test Strip

The ICA test strip was assembled from a nitrocellulose (NC) membrane, absorbent pad, and sample pad. The sialic acid-binding lectin (L-1300) and non-oxidized anti-transferrin antibody (11D3) were immobilized (8 mm from beginning on the top side of the NC membrane; 30×2.5 cm²) using a dispenser (DCI 100; Zeta Corporation, Kyunggi-do, Korea). The antibody-loaded NC membrane was dried in a chamber at 37° C. and 15% humidity for 15 min. After incubation, the absorbent pad (Grade 222; 30×2 cm²) was attached to the top and bottom side of the NC membrane with a 2 mm overlap. The combined NC membrane and absorbent pad was cut into 4-mm wide strips using a cutting machine.

Evaluation of Lectin Specificity

Three ICA test strips with loaded oxidized and non-oxidized anti-transferrin antibodies were prepared to evaluate the specificity of lectin for sTF and the results obtained by oxidizing the antibody. One pair each of the three ICA test strips loaded with oxidized and non-oxidized anti-transferrin antibodies were dipped into the sTF, β2TF, and control solution for 15 min. β2TF was prepared by reacting sTF (100 μg mL⁻¹) with a neuraminidase (10 μg mL-1) in 1×G1 reaction buffer overnight at 37° C. The sTF (1 μg mL-1), β2TF (1 μg mL⁻¹), and control solution (no protein) was prepared based on the loading buffer that contains 1×PBS with PVP (10 mg mL⁻¹) and surfactant 10G (5 mg mL⁻¹). Subsequently, the ICA test strips were dipped in the loading buffer and washed for 5 min. After washing, the ICA test strips were dipped into biotinylated sialic acid-binding lectin solution (10 μg mL-1) in the loading buffer for 15 min. The ICA test strips were then washed with the same solution for 15 min. The ICA strips were dipped into the prepared AuNP-streptavidin conjugate solution for 15 min and washed with loading. The color signal intensity was measured using the image analyzing equipment (ChemiDoc™ XRS+ Imaging System; Bio-Rad Laboratories, Hercules, Calif., USA). The captured image was analyzed using the Image Lab 4.0 software (Bio-Rad). The colorimetric signal intensities were analyzed with the profiling of the line for each strip sensor, compensating for the background signal intensity of the NC membrane. Subsequent signal intensity analyses were performed using the same method.

Determination of Effect of Sample Pretreatment

Oxidized anti-transferrin antibody-AuNP conjugate was prepared using the same method used for streptavidin, except that the blocking reagent was replaced with Neo protein saver (5 mg mL⁻¹) and the final re-suspension solution was also replaced with 10 mM borate buffer solution (pH 8.5). The ICA test strips were also prepared using the same method with the control line immobilized at 4 mm from the end on the top side of the NC membrane and the five deletion lines immobilized at 12, 14, 16, 18, and 20 mm from the end on the top side of the NC membrane. The pooled serum and CSF solutions were diluted from 20-fold to 20,000-fold with 1×PBS containing PVP (10 mg mL-1) and surfactant 10G (5 mg mL-1). For sTF and β2TF, similar concentrations of pooled serum and CSF were spiked in 1×PBS containing PVP (10 mg mL⁻¹) and surfactant 10G (5 mg mL⁻¹) based on the comparison of the titration curve results in the buffer-spiked assay. Furthermore, 100 μL of each prepared solution was applied to the prepared ICA test strips and for assays using sample pretreatment, the pooled serum and CSF were reacted with the AuNP conjugate for 15 min. In addition, the solution was purified three times by centrifugation at 13,475×g for 15 min at 10° C. and applied to the ICA test strips. After 8 min, 1×PBS containing PVP (10 mg mL⁻¹) and surfactant 10G (5 mg mL⁻¹) was applied to the sample pad, and then the results were analyzed using the method described in the previous section.

Application of Mixed Samples

The pooled serum and CSF were mixed in ratios of 1:3, 1:1, and 3:1. The mixed sample and pooled serum and CSF (20-fold dilution) were reacted with 1×AuNP conjugate for 15 min and purified three times by centrifugation at 13,475×g for 15 min at 10° C. The purified solutions were then applied to the ICA test strips and analyzed using the method previously described. For the assay using 10% and 30% serum samples, the serum was diluted with 1×PBS and the same process was repeated.

Evaluation of Clinical Samples

47 clinical samples were obtained from Neurological Surgery, P. C. Rockville Centre, N.Y., USA (13 positive and 34 negative samples). Since these samples were destined for disposal and de-identified, IRB approval was not required. An additional 13 artificially mixed samples were prepared by mixing the same volume of 13 positive samples and 13 randomly selected negative samples. The serum samples were stored at −80° C. for subsequent analysis. The clinical samples were also evaluated using the same method used for the mixed samples described above.

### Results

Detection Strategy and ICA Design

The glycan chains of β2TF in CSF have different terminal sugar residues than are found on the glycan chains of serum specific-sialo transferrin (sTF) due to the action of a brain neuraminidase. Hydrolysis of TF glycan chains by this neuraminidase removes their non-reducing terminal sialic acid residues resulting in generation of the β2TF (asialo-transferrin) glycoform. Thus, the glycan chains of CSF β2TF differ from those of sTF in which all of its glycan chains are capped with terminal sialic acid residues. After identifying a lectin with specificity for sialic acid, a detection strategy was designed for the diagnosis of CSF leakage (FIG. 1).

In the first step of the sample pretreatment process (FIG. 1A), the sample solutions are treated with an anti-transferrin (TF) antibody (Ab), specific for transferrin, which are conjugated to AuNPs and thereby capturing total TF (STF+β2TF). The pretreated sample containing TF-AuNP complexes is then loaded onto the ICA test strip (FIG. 1B). On the ICA test strip, proximal to the loading position, there are, preferably, multiple deletion lines containing sialic acid-specific lectin, and further downstream a test line, containing an anti-TF antibody, and close to the end of the test strip, preferably, a control line with anti-mouse IgG antibody that serves as a positive control. The reactions taking place in sample pre-treatment, the deletion lines, and the test line are illustrated in FIG. 1C. During the sample pretreatment process, both β2TF and sTF were captured by the anti-TF Ab-AuNP conjugates and these complexes could be recovered by centrifugation. Next, this pretreated solution containing the TF-AuNP complexes was loaded onto the ICA sample pad and then migrated along the test strip towards the absorbent pad (FIG. 1B). The specific sialic acid-binding lectin immobilized in the deletion lines selectively removed sTF-AuNP complexes but allowed the β2TF AuNP complexes to proceed along the test strip. These β2TF (asialo-transferrin)-AuNP complexes flowed forward encountering the test line where they were captured with immobilized anti-TF antibody, resulting in a detection signal at the test line. The signal intensity at the test line served as an indicator of the amount of β2TF in a given sample. The control line indicated the performance of ICA works well, and that sample had indeed flowed along the test strip.

Evaluation of the Specificity and Efficiency of Lectin in the Deletion Lines of the ICA Test Strip

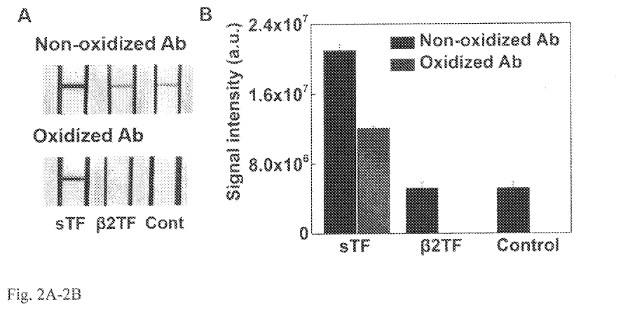

The specificity and the efficiency of the sialic acid-binding lectin in the capture of sTF at the deletion lines on the ICA test strip is critical for a successful CSF leakage diagnostic. It was not known if the specificity and the efficiency were sufficient for practical use. Hence, the specificity of sialic acid-binding lectin for sTF capture was evaluated using a NC membrane dipstick method. Anti-TF antibodies were immobilized on a NC membrane strip and the strips were dipped into the solutions containing sTF, β2TF, and PBS (negative control), respectively, followed by the sequential binding of biotinylated lectin and streptavidin-AuNP conjugate to these NC strips. This experiment confirmed the binding of sTF to the sialic acid-binding lectin. However, the results also showed weak signal for both β2TF and the control (FIG. 2A). It was hypothesized that this unexpected signal resulted from the interaction of the anti-transferrin Ab with the sialic acid-binding lectin. This lectin interaction might result from the glycan chains of antibody that also can contain terminal sialic acid residues. The mild periodate oxidation of the anti-transferrin antibody removes the terminal sialic acid residues from its glycan chains leaving aldehyde groups in these glycan chains (38). The use of oxidized antibodies (asialo-Ab) greatly reduced the undesired interaction between anti-transferrin Ab and the sialic acid-binding lectin (FIG. 2A). Fortunately, the periodate oxidation of this antibody improved the selectivity of the interaction between sialic acid-binding lectin and sTF by avoiding removing the interaction of β2TF-anti-transferrin antibody AuNP conjugate (FIG. 2B) with only a relatively small loss of sTF signal intensity.

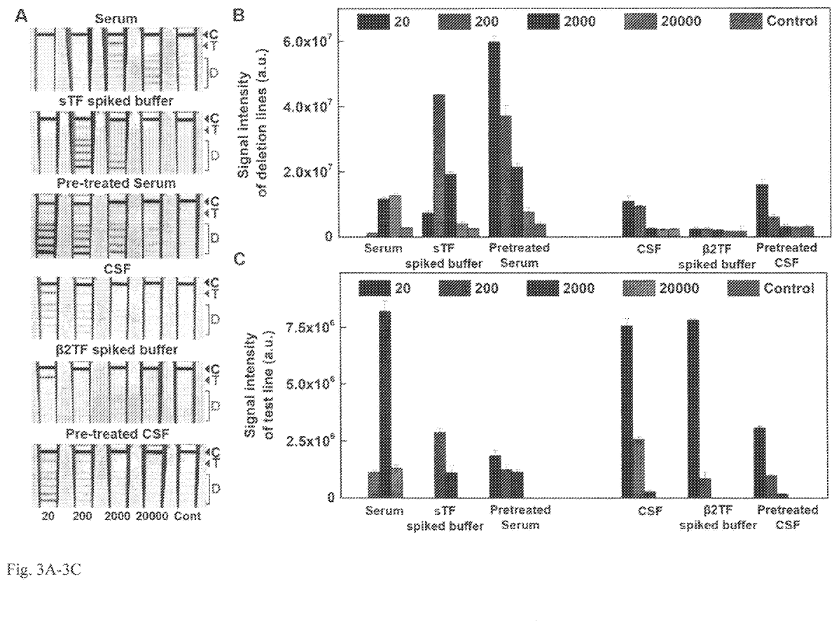

Next, the efficiency of the deletion lines in the ICA test strip were evaluated using serum and CSF. The concentrations of TF in both serum and CSF were first quantified using the ICA test strip after preparing different concentrations of TF spiked-buffer solutions. We compared the signal intensity of these TF solutions, CSF, and serum in the test lines in the ICA strip. The results showed that the concentration of TF in serum and CSF was approximately 2 mg mL⁻¹ and 20 μg mL⁻¹, respectively. Based on these concentrations we also prepared the sTF-spiked and β2TF-spiked PBS solutions and adjusted the concentration of sTF (2 mg mL⁻¹) to be comparable to that of serum and the concentration of β2TF (20 mL⁻¹) to be comparable to that of CSF. Different concentrations of the sTF-spiked, β2TF-spiked buffer solution, serum, and CSF were each applied to the ICA test strips (FIG. 3). The results showed no detectable signal in the deletion lines over the range of 20- to 200-fold diluted serum but signal was observed in the deletion lines over the range of 2,000- to 20,000-fold diluted serum. In the sTF-spiked PBS, signals were detected in the deletion lines over the range of 200- to 20,000-fold dilution. These results showed that the serum required a 20,000-fold dilution to completely remove the sTF. However, in the case of both sTF spiked in buffer and serum the deletion lines on the ICA test strip (FIG. 3A) were non-functional at 20-fold dilution due to the hook effect (36).

After quantifying the signal intensity in the five different deletion lines in each diluted sample containing sTF, the sTF-spiked PBS solutions (1 μg mL⁻¹) showed a similar pattern, that has decreasing deletion proportion from forward along to rear line, as observed for the pretreated serum samples which contained 100 μg mL⁻¹ of sTF, while the 2,000-fold diluted serum, despite which contained 1 μg mL⁻¹ of sTF, showed an inverted pattern. These results provide evidence of the presence of unidentified sialo-glycoproteins in serum that compete for the binding of TF-AuNP conjugates to the sialic acid-binding lectin immobilized in the deletion lines. Therefore, it is clear that the sample pretreatment process is required for efficient elimination of these unidentified and interfering sialo-glycoproteins. In addition, the sample pretreatment process can also eliminate the hook effect caused by excess amounts of unconjugated TF that competes with the TF-AuNP conjugates for binding to the sialic acid-binding lectin immobilized in the deletion lines, resulting in decreased signal in the deletion lines.

In the case of CSF, the lectin immobilized in the deletion lines successfully captured sTF over the entire dilution range (20- to 20,000-fold) tested, as the total protein concentration in CSF is considerably lower than that of serum (FIG. 3A). A signal for the test line could be observed for CSF over the range of 20- to 2,000 fold dilution of CSF samples; however, there was no detectable signal using 20,000 fold-diluted CSF (FIG. 3C). These results suggest that a >20,000-fold dilution of serum was required for the complete removal of sTF but that CSF solutions should be diluted <20,000-fold to detect β2TF at the test line (FIGS. 3B and C). A pretreatment process, therefore, was required to overcome the discordance between these dilution factors for serum and CSF and to eliminate the undesired interfering sialo-glycoproteins competing with sTF for the sialic acid-binding lectin immobilized in the deletion lines. The results showed that the pretreated serum and CSF solutions afforded increased signals in the deletion lines over the entire range of dilutions (FIG. 3). In addition, no hook effect was observed for the pretreated serum and CSF samples (FIG. 3A).

Evaluation of CSF Content with the ICA Strip

While the pretreatment process improves the detection sensitivity of β2TF in the test line of the ICA test strip, the determination of CSF in a test sample might still be inaccurate due to certain limitations in this assay method. First, in this assay the anti-TF Ab-AuNP conjugate binds to both sTF and β2TF simultaneously but the physiological concentration of TF in serum is approximately 100-fold higher than that in CSF. In addition, β2TF constitutes only 30% of the entire TF present in CSF with the remainder being sTF. Therefore, binding of sTF to the anti-TF Ab-AuNP conjugates should dominate when compared to β2TF. As a result, the complexes conjugated simultaneously with both of sTF and β2TF might be captured in the deletion lines. Hence, a signal for β2TF in test line might not be observed despite the presence of CSF, representing a false negative. Second, the sTF in test sample may not be completely captured at the deletion lines because of the short reaction time on the ICA test strip and/or because of the weak interaction between the sialic acid-binding lectin and glycoprotein, again resulting in a false negative result. Thus, the signal intensities both at deletion lines and a test line in an ICA test strip must be determined to overcome these limitations.

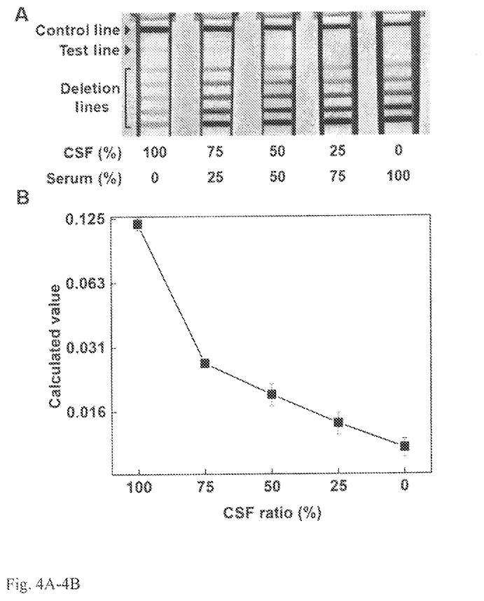

In an attempt to address these issues, different mixtures of pooled serum and CSF were prepared, and the mixtures were applied to the ICA test strips after sample pretreatment. FIG. 4A shows that as the percentage of CSF in the mixture decreased, the signal intensity at the test line decreased and, in contrast, the sum of the signal intensity in the deletion lines increased. These results suggest that the proportion of sTF and β2TF in each sample depend on the mixture ratio of CSF and serum used in a test sample. By considering the signals in both the deletion lines and test line, one could obtain a calculated value (signal intensity of the test line/the sum of signal intensities of the deletion lines). These calculated values were proportional to the content of CSF in the mixtures (FIG. 4B). In addition, mixtures of CSF and diluted serum (10% and 30% diluted in PBS) were also evaluated to mimic various body fluids, such as otorrhea, rhinorrhea and drainage from the spinal suture area. The results of these experiments showed that the calculated values also proportionally increased with an increased content of CSF. In conclusion, the use of both sample pretreatment and a calculated value that takes into account signals in both the test line and deletion lines were optimal for accurate determination of CSF leakage using the ICA test strip.

Evaluation of Clinical Samples

47 clinical samples (13 positive and 34 negative samples) obtained from brain ventricular, lumbar wound, cervical wound and postoperative drainage from spinal surgery were analyzed (Table 1).

Table 1. Comparison of evaluation with clinical sample and artificial mixture sample (AMSa) by conventional method (immunofixation) and results of immunochromatographic assay (ICA).

| | | | Results of ICA | |
|---|---|---|---|---|
| Sample | | Immuno- | | Calculated |
| No | Leaking place | fixation | Test line | value |
| 1 | Lumbar drain | Positive | Negative | Positive |
| 2 | Ventriculostomy | Positive | Positive | Positive |
| 3 | Post op lumbar drain | Negative | Negative | Negative |
| 4 | Lumbar drain | Positive | Positive | Positive |
| 5 | Post op lumbar drain | Negative | Negative | Negative |
| 6 | Brain | Positive | Positive | Positive |
| 7 | Post op lumbar drain | Negative | Negative | Negative |
| 8 | Post op drain | Negative | Negative | Negative |
| 9 | Post op drain | Negative | Negative | Negative |
| 10 | Post op drain | Negative | Negative | Negative |
| 11 | Post op drain | Negative | Negative | Negative |
| 12 | Post op drain | Negative | Negative | Negative |
| 13 | Post op drain | Negative | Negative | Negative |
| 14 | Post op drain | Negative | Negative | Negative |
| 15 | Ventriculostomy | Positive | Positive | Positive |
| 16 | Post op drain | Negative | Negative | Negative |
| 17 | Post op drain | Negative | Negative | Negative |
| 18 | Post op drain | Negative | Negative | Negative |
| 19 | Post op drain | Negative | Negative | Negative |
| 20 | Post op drain | Negative | Negative | Negative |
| 21 | Post op drain | Negative | Negative | Negative |
| 22 | Post op drain | Negative | Negative | Negative |
| 23 | Post op drain | Negative | Positive | Negative |
| 24 | Post op drain | Negative | Negative | Negative |
| 25 | Lumbar wound | Negative | Negative | Negative |
| 26 | Lumbar wound | Negative | Negative | Negative |
| 27 | Lumbar wound | Negative | Negative | Negative |
| 28 | Lumbar wound | Negative | Negative | Negative |
| 29 | Lumbar wound | Negative | Negative | Negative |
| 30 | Cervical wound | Negative | Negative | Negative |
| 31 | Brain ventric | Positive | Negative | Positive |
| 32 | Lumbar wound | Negative | Negative | Negative |
| 33 | Lumbar drain | Positive | Positive | Positive |
| 34 | Lumbar wound | Negative | Negative | Negative |
| 35 | Lumbar wound | Negative | Negative | Negative |
| 36 | Lumbar wound | Negative | Negative | Negative |
| 37 | Brain ventric | Positive | Positive | Positive |
| 38 | Brain ventric | Positive | Positive | Positive |
| 39 | Brain ventric | Positive | Positive | Positive |
| 40 | Brain ventric | Positive | Positive | Positive |
| 41 | Lumbar CSF drain | Positive | Positive | Positive |
| 42 | Lumbar wound | Negative | Negative | Negative |
| 43 | Lumbar wound | Negative | Negative | Negative |
| 44 | Lumbar wound | Negative | Positive | Negative |
| 45 | Brain ventric | Positive | Positive | Positive |
| 46 | Lumbar wound | Negative | Positive | Negative |
| 47 | Lumbar wound | Negative | Positive | Positive |
| AMS 1 | Sample 1 (positive) + | | Positive | Positive |
| | Sample 3 | | | |
| AMS 2 | Sample 2 (positive) + | | Positive | Positive |
| | Sample 5 | | | |
| AMS 3 | Sample 4 (positive) + | | Positive | Positive |
| | Sample 7 | | | |
| AMS 4 | Sample 6 (positive) + | | Positive | Positive |
| | Sample 8 | | | |
| AMS 5 | Sample 15 (positive) + | | Positive | Positive |
| | Sample 9 | | | |
| AMS 6 | Sample 31 (positive) + | | Positive | Positive |
| | Sample 18 | | | |
| AMS 7 | Sample 33 (positive) + | | Positive | Positive |
| | Sample 20 | | | |
| AMS 8 | Sample 37 (positive) + | | Positive | Positive |
| | Sample 26 | | | |
| AMS 9 | Sample 38 (positive) + | | Negative | Negative |
| | Sample 28 | | | |
| AMS 10 | Sample 39 (positive) + | | Positive | Positive |
| | Sample 32 | | | |
| AMS 11 | Sample 40 (positive) + | | Positive | Positive |
| | Sample 35 | | | |
| AMS 12 | Sample 41 (positive) + | | Positive | Positive |
| | Sample 43 | | | |
| AMS 13 | Sample 45 (positive) + | | Positive | Positive |
| | Sample 47 | | | |

^{a}AMS indicate the sample artificially mixed with same volume of positive and negative clinical sample.

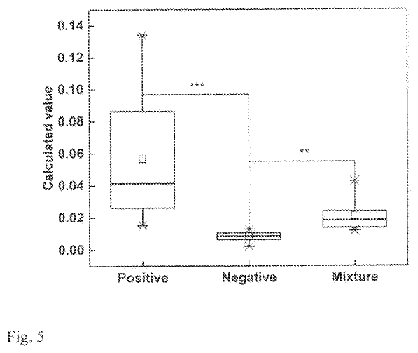

Because the positive samples contained over 90% CSF, the ICA test strips could easily discriminate positive samples, containing CSF, and negative samples, with >95% statistical significance (positive versus negative t-test; P=0.001 in FIG. 5). We next prepared 13 additional artificial positive samples by mixing positive clinical samples, containing CSF, with serum to further challenge our ICA method. The artificial positive mixtures were again clearly discriminated from the negative samples (mixture versus negative t-test; P=0.01 in FIG. 5).

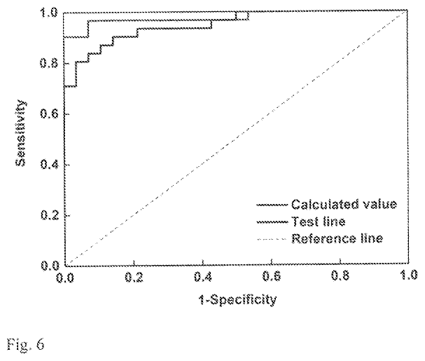

Because the number and diversity of samples were insufficient to evaluate the performance of the newly developed method, performance of the ICA method was further evaluated based on a ROC curve using two parameters (calculated value and signal intensity of the test line in FIG. 6). In these analyses the artificial mixtures were included in the positive sample group. The area under the curve (AUC) values indicated the effectiveness of the ICA test strip method in distinguishing the positive and negative samples, were 0.9728 and 0.9333 for calculated value and test line signal intensity, respectively (43). AUC values range from perfect discrimination (AUC=1) and no discrimination (AUC=0.5) between the positive and negative samples. Thus, the newly developed ICA method was significantly capable of distinguishing positive samples from negative samples. Furthermore, the Youden's index (J) was calculated from the ROC curve, which can be used for obtaining an optimal cut-off value. Based on this value, the specification of the developed method was evaluated and is summarized in Table 2. Based on the Youden's index and AUC value, it was confirmed that the calculated value was more valid than the signal intensity of the test line, although by combining the quantification of detection signals obtained from the ICA test strip with statistical analysis, we were able to determine CSF leakage with 97.1% specificity and 96.2% sensitivity.

TABLE 2
Comparison of specification of
the immunochromatographic assay (ICA)
based on parameter for determination
of cerebrospinal fluid (CSF) leakage

| Determining | Area | | | Youden's |
|---|---|---|---|---|
| parameter of CSF | under the | | | index |
| leakage | curve | Sensitivity | Specificity | (J) |
| Calculated ratio value | 0.9729 | 96.2% | 97.1% | 0.9321 |
| Signal intensity of test line | 0.9333 | 88.2% | 88.5% | 0.7669 |

### DISCUSSION

The ICA test strip containing five deletion lines and a test line was developed for detecting the presence of CSF in test samples (FIG. 1). The complete removal of sTF with a sialic acid-specific lectin immobilized in five deletion lines was best for good detection sensitivity of CSF leakage. Various unidentified sialo-glycoproteins in human fluids resulted in unacceptable false-negatives, since these sialo-glycoproteins also bind to the sialic acid-specific lectin. Thus, a sample pretreatment process was needed to eliminate interfering glycoproteins and the unconjugated TF that could compete with binding of sTF-AuNP complexes to the sialic acid-binding lectin immobilized in the deletion lines. Although this pretreatment process increased the efficiency for the sTF removal by the sialic acid-binding lectin on the five deletion lines (FIG. 3), there were several additional considerations for the accurate detection of CSF. First, the anti-TF Ab-AuNP conjugates could bind to both sTF and β2TF simultaneously. Second, the physiological concentration of sTF in the serum was approximately 100-fold higher than that in CSF. Third, β2TF constitutes only ˜30% of the total TF in the CSF. Taking these limitations into consideration, the binding of sTF to the anti-TF Ab-AuNP conjugates should dominate those of β2TF. As a result, both the sTF-AuNP and β2TF-AuNP complexes are captured in the deletion lines. Thus it was considered that the signal of β2TF in test line might not be sufficient for detection despite the presence of CSF. It was also speculated that all of the sTF in test sample might not be captured by the sialic acid-binding lectin in the deletion lines because of the short reaction time on the ICA test strip or because of the weak interaction between sialic acid-binding lectin and glycoprotein (42), resulting in the detection of a false positive signal in the test line due to sTF instead of β2TF. Finally, because the concentration of TF in test fluids from human body are variable (44-46), it was decided to measure not only the β2TF captured at a test line, but also the sTF captured with the lectin in deletion lines in order to accurately determine CSF leakage. However, when the ratio of the signal intensity of test line (for β2TF) to the signal intensity of deletion lines (sTF) was determined, the calculated values (signal of a test line divided by the signal of the sum of deletion lines) was found to be proportional to the content of CSF in test samples (FIG. 4). This resulted in high sample confidence.

In conclusion, a novel POC method and device for determining CSF leakage has been made by detecting β2TF using an ICA test strip. The effectiveness of this approach was tested on 47 clinical samples and 13 artificial mixtures prepared from positive samples and serum. It was found to discriminate between the positive and negative samples with >95% statistical significance. ROC analysis indicated that the method can be used for the determination of CSF leakage. The Youden's index of the ROC was used to define the optimal cut-off value, and the specificity and sensitivity were 100% and 90%, respectively. The test time is less than 10 minutes, with a longer sample pretreatment process (e.g. up to 60 min) which is preferred because of considerable interference caused by other sialo-glycoproteins present and the high concentration of TF in human fluids. When compared to conventional electrophoresis-based detection methods for β2TF, the entire assay time of this new method is significantly shorter.

Rapid and sensitive detection of CSF is crucial [24] to make real-time critical decisions regarding patient care. For example, if a CSF leakage occurs post-surgery, a patient may need to quickly return to the operating room to explore and repair the CSF leak, which would in turn treat the positional headaches and potential infection from contact with contaminated skin, thereby increasing the risk of developing meningitis. At the time fluid is first noticed, and if the surgeon is unsure whether the fluid contains CSF, the surgeon can often only wait for confirmatory analysis, which delays action and can lead to poorer patient prognosis. In some cases a patient might not a classic presentation of a positional headache, which can further delay the diagnosis of a CSF fluid leak. Thus, a rapid test that can detect the presence of CSF fluid would allow spine surgeons to make immediate clinical decisions leading to improved patient outcomes.

### REFERENCES

[1] Barrios, C., Ahmed, M., Arrotegui, J. I., Bjornsson, A., J Spinal Disorders 1990, 3, 205-209.
[2] Cammisa, F. P., Jr., Girardi, F. P., Sangani, P. K., Parvataneni, H. K., Cadag, S., Sandhu, H. S., Spine 2000, 25, 2663-2667.
[3] Khan, M. H., Rihn, J., Steele, G., Davis, R., Donaldson, W. F., 3rd, Kang, J. D., Lee, J. Y., Spine 2006, 31, 2609-2613.
[4] Stolke, D., Sollmann, W. P., Seifert, V., Spine 1989, 14, 56-59.
[5] Wang, J. C., Bohlman, H. H., Riew, K. D., J Bone Joint Surgery (Am) 1998, 80, 1728-1732.
[6] Eismont, F. J., Wiesel, S. W., Rothman, R. H., J Bone Joint Surgery (Am) 1981, 63, 1132-1136.
[7] Brown, K. J., Vanderver, A., Hoffman, E. P., Schiffmann, R., Hathout, Y., Int J Mass Spectrom 2012, 312, 97-106.
[8] Jou, Y. J., Lin, C. D., Lai, C. H., Chen, C. H., Kao, J. Y., Chen, S. Y., Tsai, M. H., Huang, S. H., Lin, C. W., Anal Chim Acta 2010, 681, 41-48.
[9] Whitfield, J. B., Clin Chem 2002, 48, 2095-2096.
[10] Wang, C., Li, C., Gong, W., Lou, T., Biomark Res 2013, 1, 9.
[11] Coddeville, B., Carchon, H., Jaeken, J., Briand, G., Spik, G., Glycoconj J 1998, 15, 265-273.
[12] de Jong, G., van Noort, W. L., van Eijk, H. G., Electrophoresis 1992, 13, 225-228.
[13] Kallee, E., Lohss, F., Debiasi, S., Nucl Med 1963, 2, Suppl 1:111-118.
[14] Iourin, O., Mattu, T. S., Mian, N., Keir, G., Winchester, B., Dwek, R. A., Rudd, P. M., Glycoconj J l 1996, 13, 1031-1042.
[15] Nandapalan, V., Watson, I. D., Swift, A. C., Clinical otolaryngology and allied sciences 1996, 21, 259-264.
[16] Arrer, E., Meco, C., Oberascher, G., Piotrowski, W., Albegger, K., Patsch, W., Clin Chem 2002, 48, 939-941.
[17] Vanderver, A., Schiffmann, R., Timmons, M., Kellersberger, K. A., Fabris, D., Hoffman, E. P., Maletkovic, J., Hathout, Y., Clin Chem 2005, 51, 2031-2042.
[18] Roelandse, F. W., van der Zwart, N., Didden, J. H., van Loon, J., Souverijn, J. H., Clin Chem 1998, 44, 351-353.
[19] Normansell, D. E., Stacy, E. K., Booker, C. F., Butler, T. Z., Clin Otolaryngol Allied Sci 1994, 1, 68-70.
[20] Gorogh, T., Rudolph, P., Meyer, J. E., Werner, J. A., Lippert, B. M., Maune, S., Clin Chem 2005, 51, 1704-1710.
[21] Legros, F. J., Nuyens, V., Baudoux, M., Zouaoui Boudjeltia, K., Ruelle, J. L., Colicis, J., Cantraine, F., Henry, J. P., Clin Chem 2003, 49, 440-449.
[22] Zeng, Y., Ramya, T. N., Dirksen, A., Dawson, P. E., Paulson, J. C., Nat Methods 2009, 6, 207-209.
[23] De Bank, P. A., Kellam, B., Kendall, D. A., Shakesheff, K. M., Biotechnol Bioeng 2003, 81, 800-808.
[24] Choi, D., Spann, R., Br J Neurosurg 1996, 10, 571-575.
[25] Gallo, P., Bracco, F., Morara, S., Battistin, L., Tavolato, B., J Neurol Sci 1985, 70, 81-92.

While this invention has been particularly shown and described with references to preferred embodiments thereof, it will be understood by those skilled in the art that various changes in form and details may be made therein without departing from the scope of the invention encompassed by the appended claims.

1. A method of detecting asialo-transferrin in a biological sample comprising:
a) contacting the biological sample with a first plurality of transferrin-binding antibodies conjugated to nanoparticles;
b) centrifuging the product of step a) so as to separate and obtain conjugates of transferrin bound to transferrin-binding antibodies conjugated to nanoparticles;
c) contacting the conjugates obtained in step b) with a lateral flow device, and observing if asialo-transferrin bound antibodies bind to a second plurality of transferrin-binding antibodies affixed to the lateral flow device, wherein if such antibodies bind then asialo-transferrin has been detected in the biological sample and wherein if no asialo-transferrin bound antibodies bind to the second plurality of transferrin-binding antibodies affixed to the lateral flow device then asialo-transferrin has not been detected in the biological sample,
and wherein the lateral flow device comprises in sequential order:
a portion comprising a fixed sialic acid-specific lectin; and
a portion comprising a second plurality of transferrin-binding antibodies affixed to the lateral flow device; and
a portion comprising a plurality of anti-antibody antibodies affixed to the lateral flow device.

2. The method of claim 1, wherein the first and second pluralities of transferrin-binding antibodies are IgG antibodies and/or wherein the plurality of anti-antibody antibodies is a plurality of anti-IgG antibodies.

3. The method of claim 1, wherein each antibody of the second plurality of transferrin-binding antibodies is conjugated to a nitrocellulose membrane of the lateral flow device, and/or wherein the sialic-specific lectin is affixed to a nitrocellulose membrane of the lateral flow device.

4. The method of claim 1, wherein multiple antibodies of the first plurality of transferrin-binding antibodies conjugated to nanoparticles are conjugated to the same nanoparticle.

5. The method of claim 1, wherein the nanoparticles comprise gold nanoparticles.

6. The method of claim 1, wherein the lateral flow device further comprises a fluid sample pad prior in sequential order to (i) the portion comprising a first plurality of transferrin-binding antibodies, or to (ii) the portion comprising a fixed sialic acid-specific lectin.

7. The method of claim 1, wherein the lateral flow device further comprises a fluid-absorbent pad subsequent in sequential order to the portion comprising a plurality of anti-antibody antibodies.

8. The method of claim 1, wherein sialic acid residues on glycan chains of the transferrin-binding antibodies have been oxidized.

9. The method of claim 8, wherein the transferrin-binding antibodies which have had their sialic acid residues oxidized show reduced binding to sialic acid-specific lectin compared to transferrin-binding antibodies which have not had their sialic acid residues oxidized.

10. The method of claim 9, wherein the transferrin-binding antibodies have been oxidized by treating them with a periodate.

11. The method of claim 1, wherein the portion comprising a plurality of anti-antibody antibodies affixed to the lateral flow device is a control line.

12. A kit comprising:
i) the device recited in claim 1 and
ii) a container comprising the first plurality of transferrin-binding antibodies conjugated to nanoparticles.

13. The kit of claim 12, wherein sialic acid residues on glycan chains of the first plurality of transferrin-binding antibodies have been oxidized.

14. The kit of claim 13, wherein the transferrin-binding antibodies which have had their sialic acid residues oxidized show reduced binding to sialic acid-specific lectin compared to transferrin-binding antibodies which have not had their sialic acid residues oxidized.

15. The kit of claim 12, wherein the nanoparticles comprise gold nanoparticles.

16. A method comprising:
performing surgery on the central nervous system of a subject;
obtaining one or more samples of the subject's blood, wherein if more than one sample is obtained then the samples are obtained at different time points during the surgery; and
detecting if cerebrospinal fluid has leaked into the blood of the subject during surgery comprising the method of claim 1 on one or more blood samples and observing if asialo-transferrin bound antibodies bind to the second plurality of transferrin-binding antibodies, wherein if such antibodies bind then cerebrospinal has been detected in the biological sample and wherein if no asialo-transferrin bound antibodies bind to the second plurality of transferrin-binding antibodies then cerebrospinal has not been detected in the biological sample.

17. The method claim 16, wherein the sample is a serum sample, an otorrhea sample, a rhinorrhea sample, or comprises drainage from a spinal suture area.

18. The method claim 16 or 17, wherein centrifuging the product of step a) so as to separate and obtain conjugates of transferrin bound to transferrin-binding antibodies conjugated to nanoparticles separates the conjugates from all, or substantially all, non-transferrin sialyated glycoproteins from the sample.

19. The method of claim 16, further comprising oxidizing sialic acid residues on glycan chains of the first plurality of transferrin-binding antibodies prior to contacting with the sample.

20. A lateral flow device comprising, in sequential order, a portion comprising a fixed sialic acid-specific lectin; a portion comprising a plurality of transferrin-binding antibodies affixed to the lateral flow device; and a portion comprising a plurality of anti-antibody antibodies affixed to the lateral flow device.

21-28. (canceled)
